(19) United States
(12) Patent Application Publication
(10) Pub. No.: US 2020/0289716 A1
(43) Pub. Date: Sep. 17, 2020

(54) BIOCOMPATIBLE COMPOSITE MATERIAL FOR INSERTION INTO A HUMAN BODY

(71) Applicant: CARL FREUDENBERG KG, Weinheim (DE)

(72) Inventors: Karl-Heinz Heffels, Pfungstadt (DE); Denis Reibel, Herrlisheim (FR); Dirk Grafahrend, Mannheim (DE); Alexandra Kaul, Hemsbach (DE); Yosuke Kadomae, Koga-shi, Ibaraki (JP); Guenter Germann, Heidelberg (DE)

(21) Appl. No.: 16/758,861

(22) PCT Filed: Oct. 26, 2018
(86) PCT No.: PCT/EP2018/079394

§ 371 Date: Apr. 24, 2020

(30) Foreign Application Priority Data
Oct. 26, 2017 (DE) 10 2017 009 989.8

(51) Int. Cl. A61L 29/14; A61F 2/20; A61L 29/12; A61L 29/04; A61L 29/16; A61L 29/08; A61L 29/06

(52) U.S. Cl. CPC A61L 29/148; A61F 2/20; A61L 29/146; A61L 29/126; A61L 29/045; A61L 29/046; A61L 2420/02; A61L 29/16; A61L 29/085; A61L 29/06; A61F 2210/0076; A61F 2240/001; A61L 29/048

(57) ABSTRACT

A biocompatible composite material for complete or partial insertion into a human body includes at least one layer comprising an elastomeric material, and at least one textile fabric arranged on the at least one layer comprising the elastomeric material. The at least one textile fabric forms a surface of the biocompatible composite material. The at least one textile fabric includes bioresorbable fibers that are embedded at least partially in the at least one layer comprising the elastomeric material.

### CROSS-REFERENCE TO PRIOR APPLICATIONS

This application is a U.S. National Phase Application under 35 U.S.C. §371 of International Application No. PCT/EP2018/079394, filed on Oct. 26, 2018, and claims benefit to German Patent Application No. DE 10 2017 009 989.8, filed on Oct. 26, 2017. The International Application was published in German on May 2, 2019 as WO 2019/081700 under PCT Article 21(2).

### FIELD

The present invention relates to a composite material for complete or partial insertion into a human body and in particular for implantation in a human body. The invention further relates to a method for producing the composite material and to the use thereof for producing a medical device for the complete or partial insertion into a human body and in particular an implant. High requirements, for example good biocompatibility, are placed on materials which are to be introduced into a human body. Biocompatibility refers to the property of materials in a biological environment to perform their predetermined functions adapted to the situation while at the same time the host body shows an acceptable reaction to the material. This is verified for medical devices within the scope of their approval according to the DIN EN ISO 10993 standard. Hereinafter, “biocompatible” materials shall refer to materials that have passed the test according to DIN EN ISO 10993 (year).

### BACKGROUND

Particularly high requirements are placed on materials which are to remain permanently in the human body. For example, implants must meet high requirements since they are to remain in the human body permanently or at least for a period of a few days as materials implanted in the body. Medical implants have the task of supporting or replacing bodily functions, while with plastic implants the shape of possibly destroyed body parts is to be restored or changed.

Although the silicone often used in implants is basically biocompatible, there are occasionally still undesired immune reactions. The host body's immune system is activated upon implantation and attempts to resorb the foreign material. If the immune cells do not achieve resorption on account of the foreign material's properties, the body starts to envelop the implant with a fibrous sleeve and thereby separate it from the surrounding tissue. This separation becomes a problem at least when the scar tissue capsule hardens and leads to deformations of the surrounding tissue.

It is known that surface and structure of an implant are critical to how the host body will handle the implant. Structured surfaces exhibit higher acceptance in the host bodies with less appearance of the capsule formations described above (US 2012/0209381 structured surface less capsule contraction). The disadvantage of the commonly used structured materials is that they do not allow for direct interaction of endogenous tissue with the implant, so that these are not fixed 100% at the implantation site.

### SUMMARY

In an embodiment, the present invention provides a biocompatible composite material for complete or partial insertion into a human body. The biocompatible composite material includes at least one layer comprising an elastomeric material, and at least one textile fabric arranged on the at least one layer comprising the elastomeric material. The at least one textile fabric forms a surface of the biocompatible composite material. The at least one textile fabric includes bioresorbable fibers that are embedded at least partially in the at least one layer comprising the elastomeric material.

### BRIEF DESCRIPTION OF THE DRAWINGS

Embodiments of the present invention will be described in even greater detail below based on the exemplary figures. The present invention is not limited to the exemplary embodiments. All features described and/or illustrated herein can be used alone or combined in different combinations in embodiments of the present invention. The features and advantages of various embodiments of the present invention will become apparent by reading the following detailed description with reference to the attached drawings which illustrate the following:

FIG. 1: Result of tensile test of pure silicone layer as reference.

FIG. 2: Result of tensile test of the composite material from Example 1.

FIG. 3: Micrograph of the surface of the composite material from Example 1 after two weeks of storage at 37° C. in PBS.

FIG. 4: Schematic cross section of a composite material according to the invention.

FIG. 5: Schematic cross section of a composite material according to the invention in its configuration as a catheter.

FIG. 6: Micrograph of the sectional view of a composite material according to the invention.

### DETAILED DESCRIPTION

One approach is to employ biocompatible materials for the surface of implants which can interact with the host body. These can be bioresorbable materials which can be decomposed and metabolized or excreted by endogenous cells. If these materials are designed as support structures, cells can migrate into these structures in order to build up new endogenous tissue. The support structure material is resorbed during this process.

There are currently no products on the market that pursue this approach. Presumably, this is because the implants would lose their function in the course of resorption.

In an embodiment, the present invention provides a composite material for the complete or partial insertion into a human body which at least partially overcomes the aforementioned disadvantages and is well accepted by the immune system in particular when inserted into the human body and has good long-term stability.

These advantages are achieved according to an embodiment of the present invention by a biocompatible composite material for complete or partial insertion into a human body, comprising at least one layer comprising an elastomeric material, and at least one textile fabric arranged on said layer and forming the surface of the composite material, said textile fabric having bioresorbable fibers which are at least partially embedded in the layer of elastomeric material.

In the composite material according to an embodiment of the invention, the connection between textile fabric and elastomeric material can be imparted via the bioresorbable fibers, which are at least partially embedded in the layer of elastomeric material.

This embedding can be obtained, for example, by applying the textile fabric to an elastomeric precursor material, for example an unvulcanized silicone layer, and pressing it into the latter. Impressing has the purpose of introducing the fibers of the textile fabric into the precursor layer. The composite can be subsequently solidified, for example by vulcanization of the precursor to form the elastomeric material, and cured in its elastomeric portion.

By embedding the bioresorbable fibers in the layer of elastomeric material, a stable composite material with high layer adhesion can be obtained. High layer adhesion means that the composite material can be handled in the usual way and can be inserted into the human body, for example, without the adhesion between elastomeric material and textile fabric coming loose.

Moreover, the composite material according to an embodiment of the invention offers a plurality of further advantages when fully or partially inserted into a human body.

An advantage of the composite material is that it has a surface formed of a textile fabric having bioresorbable fibers, since this allows for a biocompatibility-increasing interaction with the surrounding tissue. Due to their fiber structure, textile fabrics have a three-dimensional structuring. As discussed above, structured surfaces can minimize the incidence of unwanted immune responses which means that such surface is perfectly suited for implants and other medical devices that interact with the body as a biological system. Nonwoven fabrics are particularly preferred, since the fibers are present as vertebrae and have a strong three-dimensional structuring.

A possible measure of the form of the three-dimensional structuring of the surface is the mean pore size of the textile fabric.

Preferably, the textile fabric has an average pore size of from 50 to 300 μm, preferably from 70 to 250 μm, more preferably from 100 to 200 μm. The pore size is measured before insertion into the elastomeric material. Measuring takes place in accordance with ASTM E 1294 (1989).

The bioresorbable fibers can be resorbed over time after insertion into the body. Here, it is advantageous that the bioresorbable fibers are also present within the elastomeric layer since cavities are formed in the layer of elastomeric material during bioresorption, comparable to a dynamically changing, three-dimensional structuring on the surface of the composite material. Over time, the layer of elastomeric material is thus provided with cavities. Generally, formation of the cavities takes place continuously, wherein more than half, more preferably more than 75 wt %, in particular more than 90 wt % of the textile fabric are resorbed after 60 days. As a result, the layer of elastomeric material becomes successively the surface layer of the composite material which can be thereby imparted with a permanent structuring having the aforementioned advantages. The dynamically changing surface offers, already during resorption, a three-dimensional environment to the endogenous cells which can be populated by them and rebuilt by normal wound healing processes. This way, the composite material according to the invention enables the ingrowth of body tissue and hence a stepwise replacement of the textile fabric by endogenous tissue.

A further advantage of the composite material according to an embodiment of the invention is that, at least during the initial period after insertion into the body, the surface of the elastomeric material in the body can be separated from the tissue by the bioresorbable coating which will increase its acceptance and tissue compatibility after implantation.

Moreover, the composite material of an embodiment the invention is characterized in that it can have excellent elasticity due to the use of an elastomeric material. As a result, good adaptation to deforming forces can be ensured outside and inside the body. The high elasticity is particularly advantageous if the composite material is to be introduced for example as an implant into the body through an as small as possible body opening. Its high elasticity allows the composite material to deform intensely, for example to be elongated in order to be able to be inserted through the small body opening.

In a preferred embodiment of the invention, the composite material is characterized by an elasticity measured according to DIN 53504 S2 at a rate of 200 mm/min from 50% to 500%, preferably from 200% to 500%, more preferably from 400% to 500%. It was surprising for a person skilled in the art that the composite material according to the invention can have such high elasticity. In particular, it was to be expected that delamination of the coating will occur during tensile stress. The fact that this can be avoided is probably due to the high layer adhesion of the composite material according to the invention.

The longer the time that the composite material is to remain in the human body, the stronger the advantageous effects are.

Naturally, the effects caused by the three-dimensionally structured surface come to bear even more, the larger the proportion of the textile fabric at the surface of the implant is. Thus, in an advantageous embodiment of the invention, the proportion of the textile fabric at the surface of the composite material is more than 50%, more preferably more than 70%, even more preferably more than 90% and in particular 100%. The aforementioned values relate to the state before insertion into the human body.

The bioresorbable fibers may comprise a wide variety of fiber materials. Preferably, the fibers comprise bioresorbable fiber materials selected from the group consisting of natural polymers, proteins, peptides, sugars, chitosan, chitin, gelatin, collagen, polyvinyl alcohol, polyvinylpyrrolidone, dextran, pullulan, hyaluronic acid, polycapolactones, polylactides, polyglycolides, polyhydroxyalkanolates, polydioxanones, polyhydroxybutyrates, polyanhydrides, polyphosphoric esters, polyesteramides and mixtures and copolymers thereof, and/or consist at least 70 wt % and/or at least 80 wt % and/or at least 90 wt % and/or at least 95 wt % of them, based in each case on the total weight of the bioresorbable fibers.

In a further embodiment of the invention, the fiber material consists entirely of the aforementioned materials, wherein customary auxiliaries, for example catalyst residues, may also be present in the fiber material. In a particularly preferred embodiment of the invention, the fibers only comprise gelatins as bioresorbable fibrous material and/or consist at least 70 wt % and/or at least 80 wt % and/or at least 90 wt % and/or at least 95 wt % of gelatin, based in each case on the total weight of the bioresorbable fibers. According to the invention, porcine gelatin is preferred because it is not a transmitter of bovine spongiform encephalopathy (BSE). In addition, the bioresorbable fibers usually contain water. For example in an amount of 1 wt % to 15 wt %.

In a further preferred embodiment of the invention, the bioresorbable fibers additionally contain at least one hydrophilic additive. Preferably, it is also bioresorbable. Preferably, the hydrophilic additive is selected from the group consisting of: Carbomer [9003-01-4], acetic acid ethenyl ester, polymer with 1-ethenyl-2-pyrrolidinone [25086-89-9], 1-ethenyl-2-pyrrolidinone homopolymer [9003-39-8], cellulose hydroxypropyl methyl ether [9004-65-3], polycarbophile [9003-97-8], 1-ethenyl-2-pyrrolidinone homopolymer [9003-39-8], methyl cellulose (E 461), ethyl cellulose (E 462), hydroxypropyl cellulose (E 463), hydroxypropyl methyl cellulose (E 464), methyl ethyl cellulose (E 465), sodium carboxy methyl cellulose (E 466), hydroxyethyl cellulose, hydroxybutyl methyl cellulose, cellulose glycolate=carboxymethyl cellulose, cellulose acetate (e.g. available from Chisso, Eastman), cellulose acetate butyrate (e.g. available from Eastman, FMC), cellulose acetate maleate, cellulose acetate phthalate (e.g. available from Eastman, FMC, Parmetier), cellulose acetate trimellitate (e.g. available from Eastman, Parmetier), cellulose fatty acid esters (cellulose dilaurate, cellulose dipalmitate, cellulose distearate, cellulose monopalmitate, cellulose monostearate, cellulose trilaurate, cellulose tripalmitate, cellulose tristearate, agar [9002-18-0], alginic acid [9005-32-7], ammonium alginate [9005-34-9], calcium alginate [9005-35-0], cellulose, carboxymethyl ether, calcium salt [9050-04-8], cellulose, carboxymethyl ether, sodium salt [9004-32-4], carrageenan [9000-07-1], carrageenan [9062-07-1], carrageenan [11114-20-8], carrageenan [9064-57-7], cellulose [9004-34-6], carob rubber [9000-40-2], corn starch and pregelatinized starch, dextrine [9004-53-9], cellulose, 2-hydroxyethyl ether [9004-62-0], hydroxyethyl methyl cellulose [9032-42-2], cellulose, 2-hydroxypropyl ether [9004-64-2], cellulose, 2-hydroxypropyl ether (low substituted) [9004-64-2], hydroxypropyl starch [113894-92-1], ethenol, homopolymer [9002-89-5], potassium alginate [9005-36-1], sodium hyaluronate [9067-32-7], starch [9005-25-8], pregelatinized starch [9005-25-8], polyethylene oxide, polyethylene glycol. The aforementioned hydrophilic additives are present, for example, in an amount of 0.1 wt % to 30 wt %, preferably from 0.5% to 20%, more preferably from 1% to 10%, based in each case on the total weight of the bioresorbable fibers. Sodium hyaluronate, hyaluronic acid, polyethylene oxide and polyethylene glycol are particularly preferred according to the invention.

The advantage of using the hydrophilic additives is that they can achieve a particularly high initial wettability of, for example, less than 10 seconds, preferably less than 5 seconds, more preferably less than 2 seconds. The high initial wettability is advantageous in order to be able to soak the textile fabric with active ingredient solutions before the composite material is inserted into the human body.

In particular as regards the use of the composite material according to the invention in the human body, it may be, in particular, useful if one or more medicaments selected from the group consisting of antimicrobial agents, anesthetics, anti-inflammatory agents, anti-scar agents, antifibrotic agents, chemotherapeutic agents and leukotriene inhibitors are present in and/or on the bioresorbable fibers. Antimicrobial substances and/or antibiotics are particularly suitable for preventing infection.

The bioresorbable fibers can be continuous filaments or staple fibers, continuous filaments being fibers with theoretically unlimited length and staple fibers being fibers with limited length. In a preferred embodiment of the invention, the bioresorbable fibers are designed as continuous filaments and/or staple fibers having a minimum length of 5 mm, for example from 5 mm to 10 cm. In practical tests it has been found that such long fibers can penetrate particularly well into the layer of elastomeric material.

In a further preferred embodiment of the invention, the textile fabric has a surface weight of from 10 to 300 g/m², preferably from 50 to 200 g/m², more preferably from 70 to 150 g/m². This has proven to be advantageous since a textile fabric with such surface weights has sufficient stability in order to be able to be applied without creases to the very wide variety of layers of elastomeric material of three-dimensional geometry.

Moreover, a textile fabric with good mechanical strength can be obtained by means of the aforementioned surface weights. For example, a maximum tensile force of at least 0.5 to 100 N, preferably of 1.0 to 50 N, more preferably of 2.0 to 30 N can be imparted to the textile fabric measured with a width of 20 mm. This is advantageous since a minimum maximum tensile force is required for processing the textile fabric.

The period of time in which the textile fabric is resorbed depends on various parameters, including inter alia the thickness of the textile fabric. Against this background, it has proven to be advantageous in most cases to design the textile fabric with an average thickness of less than 2 mm, preferably from 5 to 700 nm.

The textile fabric can basically comprise one or more fibrous layers. Particularly preferably, it comprises only one fiber layer since adhesion problems that often occur between a plurality of fiber layers can be avoided.

The textile fabric can also be present in a wide variety of embodiments, for example as woven fabric, knitted fabric or nonwoven fabric. Nonwovens, as set forth above, are particularly preferred according to the invention, in particular nonwovens produced in a rotary spinning process. In rotary spinning processes, nonwovens can be produced, for example, by providing a fluid containing fibrous material which can be present as a melt, solution, dispersion or suspension, the fluid being spun, drawn and deposited as a nonwoven by rotary spinning. With this technique, work can be carried out at low temperatures up to 60° C. This enables particularly gentle processing of the biopolymers and active ingredients.

Nonwovens particularly preferred according to the invention are nonwovens as described in WO 2008/107126A1, WO 2009/036958 A1, EP 2 409 718 A1, EP 2 042 199 A1, EP2129339B1, CA2682190C. The aforementioned publications are incorporated by reference into the present invention.

The layer of elastomeric material may have a very wide variety of elastomeric materials. Silicone elastomers, especially medical grade silicone elastomers, are particularly preferred because they are relatively inert and do not react with the body.

Preferably, the layer of elastomeric material consists at least 70 wt % and/or at least 90 wt % and/or at least 95 wt % of the aforementioned silicone elastomers. Most preferably, the layer of elastomeric material consists 100 wt % of medical grade silicone elastomers, wherein customary additives may be present.

The thickness of the layer comprising the elastomeric material may vary depending on the materials employed and the targeted use.

Thicknesses in the range from 100 μm to 5000 μm, preferably from 100 μm to 4000 μm, more preferably from 100 μm to 3000 μm have proven to be generally favorable. The layer of elastomeric material can basically comprise one or more layers.

In one embodiment of the invention, the composite material has a carrier layer. It is preferably arranged on the side of the layer facing away from the textile fabric, comprising the elastomeric material. The carrier layer preferably consists of a biocompatible material since it can remain in the composite material and meets the requirements when inserted into the human body. This is why the carrier layer preferably consists of an elastomeric material, in particular of silicone. It is also conceivable to use other carrier layers, for example films, plates or molded bodies.

A further subject matter of an embodiment of the present invention is the formation of the composite material as a medical device for complete or partial insertion into the human body, in particular as an implant, for example as a voice prosthesis and/or for body access, for example as a catheter or fistula adapter. An implant is to be understood as meaning a material that is implanted in the body and intended to remain there permanently or at least for a period of time, e.g. a few days up to 10 years.

In a particularly preferred embodiment of the invention, the composite material is designed as a medical device for body access, in particular as a catheter, and has the following features:
the layer of elastomeric material is designed as a sleeve,
the textile fabric having bioresorbable fibers is arranged as a coating on the outside of the sleeve.

With implants of this type, the entire surface can be formed by the coating so that the aforementioned advantages can be utilized particularly efficiently. It is therefore preferred according to the invention that in this embodiment, the coating covers the outside of the sleeve completely.

The soft tissue implant is suitably shaped in such a way that it can fill a cavity in the human body according to shape and size.

In a preferred embodiment of the invention, the composite material according to the invention can be produced by a method comprising the steps of:
1. Providing a carrier layer;
2. Applying a biocompatible elastomeric precursor material, in particular of unvulcanized silicone, to one side of the carrier layer;
3. Applying a textile fabric having bioresorbable fibers to the elastomeric precursor material such that the fibers of the textile fabric penetrate the elastomeric precursor material at least partially;
4. Crosslinking the elastomeric precursor material to an elastomeric material.

The first method step comprises the provision of a carrier layer. A biocompatible material is preferably used as the carrier layer since it can remain in the composite material and meets the requirements when inserted into the human body. This is why the carrier layer preferably consists of an elastomeric material, in particular of silicone. It is also conceivable to use other carrier layers, for example films or moldings.

The second method step comprises applying a biocompatible elastomeric precursor material, in particular unvulcanized silicone, to one side of the carrier layer. The most varied materials, such as unvulcanized and/or incompletely vulcanized silicone, can be used as elastomeric precursor material. These materials can be converted to elastomeric materials by crosslinking in the form of vulcanization. When using silicone in the carrier layer and an elastomeric silicone precursor material, it is advantageous that a particularly homogeneous bond is formed between the layers since then both layers have the same properties.

The third method step comprises applying a textile fabric having bioresorbable fibers to the elastomeric precursor material such that the fibers of the textile fabric penetrate the elastomeric precursor material at least partially. Penetration of the fibers of the textile fabric into the elastomeric precursor material can be accomplished, for example, by pressurizing the composite of textile fabric and elastomeric precursor material. To this end, the elastomeric precursor material preferably has a viscosity of 200 mPa*s to 4000 mPa*s, more preferably of 300 mPa*s to 3000 mPa*s and in particular of 500 mPa*s to 2000 mPa*s. The aforementioned textile fabrics are preferable for use as the textile fabric. Particular preference is given to fabrics that comprise fibers made of gelatin.

The fourth method step comprises crosslinking the elastomeric precursor material to an elastomeric material. When using silicone precursor materials, crosslinking can be carried out in a simple manner by heating (vulcanization). It was surprising for a person skilled in the art that crosslinking also works in the presence of a gelatinous textile fabric since gelatin is known to have a multiplicity of functional groups. The latter are known as catalyst poison to the person skilled in the art.

It is conceivable to remove the carrier layer after the crosslinking step. However, when using a biocompatible carrier layer it is preferred if it remains in the composite material. As already explained above, the composite material according to the invention is perfectly suited for use as a medical device for complete or partial insertion into the human body.

A further subject matter of an embodiment of the present invention is therefore to use the composite material according to the invention for producing a medical device for complete or partial insertion into the human body, in particular as an implant, for example as a voice prosthesis and/or for body access, for example as a catheter or fistula adapter.

In a further embodiment of the invention this also comprises the medical device itself

Embodiment of the invention are explained in more detail below with reference to an example:

### EXAMPLE

### Preparation of a Composite Material According to the Invention

The following starting materials are used for producing an elastomeric precursor material: MED-6400A (component A) and MED-6400B (component B) NuSil technology. Components A and B are mixed at a weight ratio of 1:1 at room temperature. The mixture is further processed without bubbles. The elastomeric precursor material thus obtained is cast onto the surface of a POM plate as carrier layer having a surface area of 225 cm². The plate coated with elastomeric precursor material is kept horizontal for 30 minutes to level and evaporate the solvent. A gelatin nonwoven is then applied to the surface of the coated plate. The composite of gelatin nonwoven and silicone-coated POM plate is produced by crosslinking the elastomeric precursor. For this purpose, it is treated in a programmable oven with the following temperature program: 30 Minutes at room temperature, 45 minutes at 75° C. and 135 minutes at 150° C. with constant change (programming). After cooling of the cured sample, a silicone/gelatin composite nonwoven is obtained by peeling off the POM plate. Tensile tests are carried out for the obtained composite material according to the invention with a tensile testing machine pursuant to DIN 53504 S2 at a head speed of 200 mm/min.

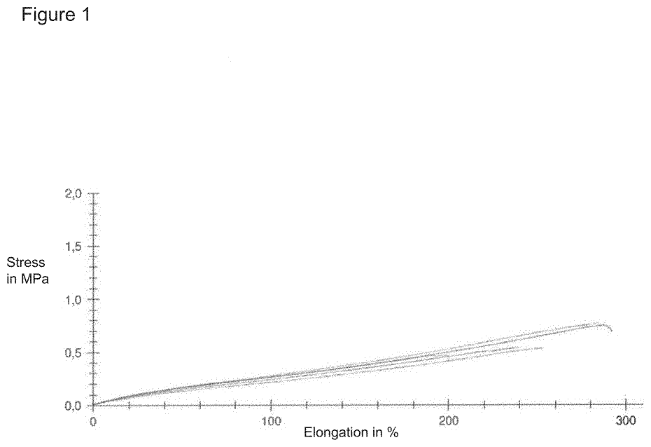

FIG. 1 shows the result of a tensile test with a pure silicone layer as reference. The tensile stress curve is linear, which is typical of elastomers. With maximum voltages of 0.4 MPa to 0.7 MPa at a maximum elongation of 200% to 300%.

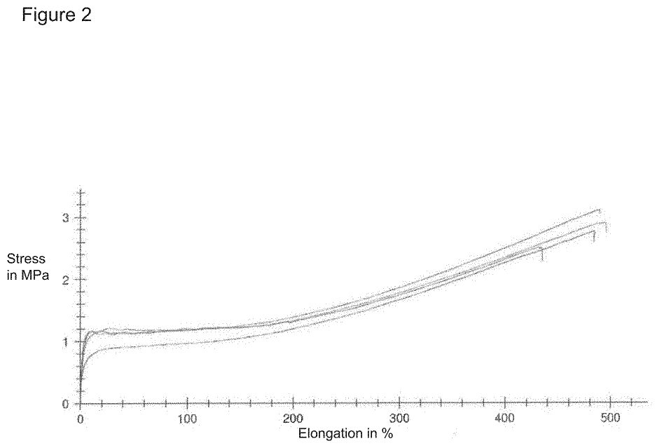

FIG. 2 shows the result of a tensile test of the composite material of Example 1. The gelatin nonwoven in the composite brings about a high stress absorption of approximately 1 MPa with minor elongation of 10-20%. The maximum elongation (HDZ) at 400%-500% has almost doubled compared to the pure silicone layer, presumably because the fibers tear independently of the elastomer and hold it together longer. From 200% elongation, stress absorption starts to increase linearly again. In this range, the elastomer portion probably absorbs the applied forces whereas before, up to 200% elongation, the forces were absorbed by the nonwoven. The maximum tensile force (HZK) of 2.4 to 3 MPa in this composite is five times greater than with a pure silicone layer.

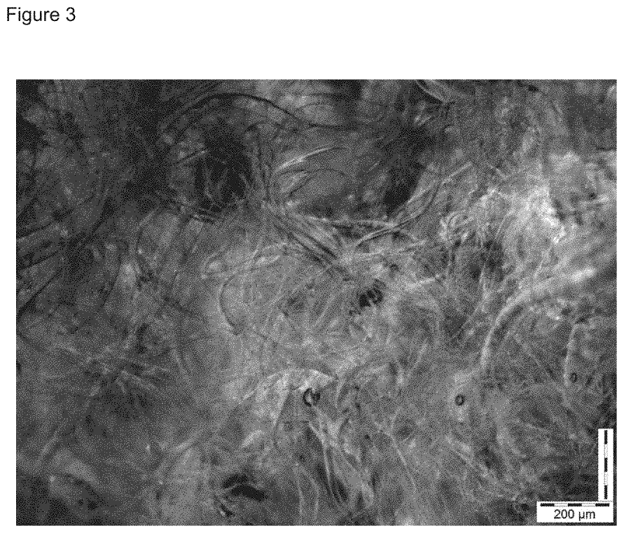

FIG. 3 shows a micrograph of the composite nonwoven surface of Example 1 after two weeks storage at 37° C. in PBS. The crosslinked fibers are still visible at this time.

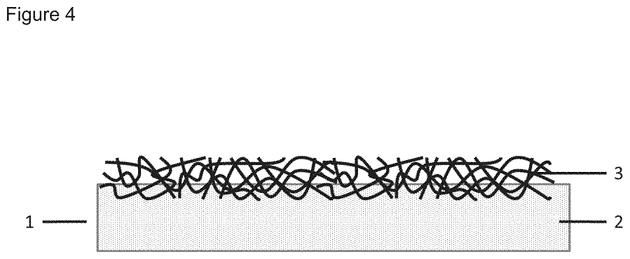

FIG. 4 shows the schematic cross section of a composite material (1) according to the invention comprising a layer (2) consisting of an elastomeric material, and a textile fabric (3) arranged on this layer (2) and forming the surface of the composite material, the textile fabric (3) having bioresorbable fibers that are at least partially embedded in the layer (2) of elastomeric material.

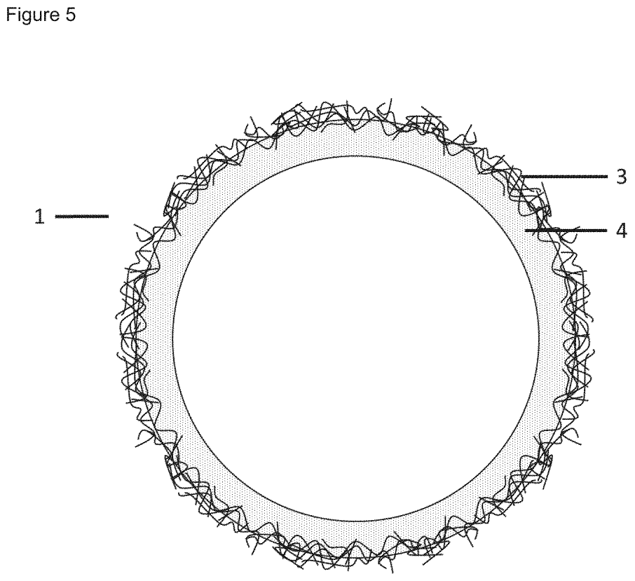

FIG. 5 shows the schematic cross section of a composite material (1) according to the invention in its embodiment as a catheter. The catheter has a sleeve (4) made of elastomeric material, here made of silicone. On the outside of the sleeve (4), the catheter has a textile fabric (3) having bioresorbable fibers, wherein the bioresorbable fibers penetrate the sleeve (4) of elastomeric material at least partially.

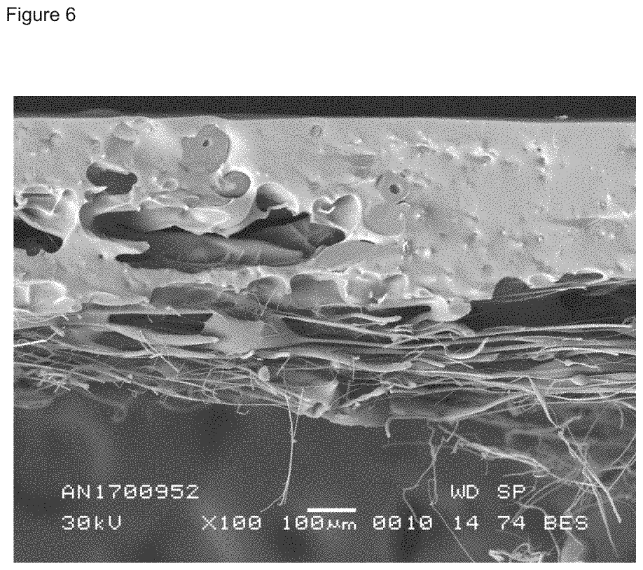

FIG. 6 shows an electron micrograph of the sectional view of a composite material according to the invention. A gelatin nonwoven is arranged as a textile fabric on the layer of elastomeric material, here silicone. One clearly recognizes how the fibers of the gelatin nonwoven penetrate the layer of silicone.

While embodiments of the invention have been illustrated and described in detail in the drawings and foregoing description, such illustration and description are to be considered illustrative or exemplary and not restrictive. It will be understood that changes and modifications may be made by those of ordinary skill within the scope of the following claims. In particular, the present invention covers further embodiments with any combination of features from different embodiments described above and below. Additionally, statements made herein characterizing the invention refer to an embodiment of the invention and not necessarily all embodiments.

The terms used in the claims should be construed to have the broadest reasonable interpretation consistent with the foregoing description. For example, the use of the article “a” or “the” in introducing an element should not be interpreted as being exclusive of a plurality of elements. Likewise, the recitation of “or” should be interpreted as being inclusive, such that the recitation of “A or B” is not exclusive of “A and B,” unless it is clear from the context or the foregoing description that only one of A and B is intended. Further, the recitation of “at least one of A, B and C” should be interpreted as one or more of a group of elements consisting of A, B and C, and should not be interpreted as requiring at least one of each of the listed elements A, B and C, regardless of whether A, B and C are related as categories or otherwise. Moreover, the recitation of “A, B and/or C” or “at least one of A, B or C” should be interpreted as including any singular entity from the listed elements, e.g., A, any subset from the listed elements, e.g., A and B, or the entire list of elements A, B and C.

1. A biocompatible composite material for complete or partial insertion into a human body, the biocompatible composite material comprising:
at least one layer comprising an elastomeric material and
at least one textile fabric arranged on the at least one layer comprising the elastomeric material and forming a surface of the biocompatible composite material,
wherein the at least one textile fabric includes bioresorbable fibers that are embedded at least partially in the at least one layer comprising the elastomeric material.

2. The biocompatible composite material according to claim 1, wherein the embedding of the bioresorbable fibers into the at least one layer comprising the elastomeric material has been obtained by applying the at least one textile fabric to an elastomeric precursor material and impressing the at least one textile fabric in the elastomeric precursor material.

3. The biocompatible composite material according to claim 1, wherein the at least one textile fabric is a nonwoven fabric.

4. The biocompatible composite material according to claim 1, wherein the at least one textile fabric has a mean pore size of from 50 to 300 μm.

5. The biocompatible composite material according to claim 1, wherein, after insertion into the human body, cavities are formed in the at least one layer comprising the elastomeric material over time due to bioresorption of the at least one textile fabric.

6. The biocompatible composite material according to claim 1, having an elasticity measured according to DIN 53504 S2 at a speed of 200 mm/min of from 50% to 500%.

7. The biocompatible composite material according to claim 1, wherein a proportion of the at least one textile fabric at the surface of the biocompatible composite material is more than 50%.

8. The biocompatible composite material according to claim 1, wherein the bioresorbable fibers comprise bioresorbable fiber materials selected from the group consisting of natural polymers, proteins, peptides, sugars, chitosan, chitin, gelati, collagen, polyvinyl alcohol, polyvinylpyrridone, dextran, pullulan, hyaluronic acid, polycapolactones, polylactides, polyglycolides, polyhydroxyalkanolates, polydioxanones, polyhydroxybutyrates, polyanhydrides, polyphosphoric esters, polyesteramides and mixtures and copolymers thereof and/or consist at least 70 wt % and/or at least 80 wt % and/or at least 90 wt % and/or at least 95 wt % of them, in each case based on the total weight of the bioresorbable fibers.

9. The biocompatible composite material according to claim 1, wherein one or more medicaments selected from the group consisting of antimicrobial agents, anesthetics, anti-inflammatory agents, anti-scar agents, antifibrotic agents, chemotherapeutic agents and leukotriene inhibitors are present in and/or on the bioresorbable fibers.

10. The biocompatible composite material according to claim 1, wherein the bioresorbable fibers are configured as continuous filaments and/or staple fibers having a minimum length of 5 mm.

11. The biocompatible composite material according to claim 1, wherein the at least one textile fabric is a nonwoven fabric produced in a rotary spinning process.

12. The biocompatible composite material according to claim 1, wherein the at least one layer comprising the elastomeric material comprises silicone elastomers.

13. The biocompatible composite material according to claim 1, configured as a medical device for body access having the following features:
a. the at least one layer comprising the elastomeric material is configured as a sleeve, and
b. the at least one textile fabric having the bioresorbable fibers is arranged as a coating on an outside of the sleeve.

14. A method for producing the biocompatible composite material according to claim 1, the method comprising:
a. providing a carrier layer;
b. applying a biocompatible elastomeric precursor material to one side of the carrier layer;
c. applying the at least one textile fabric having the bioresorbable fibers to the elastomeric precursor material such that the fibers of the textile fabric at least partially penetrate the elastomeric precursor material; and
d. crosslinking the elastomeric precursor material to form the elastomeric material.

15. A method of producing a medical device for complete or partial insertion into the human body with the biocompatible composite material according to claim 1, the method comprising:
forming the medical device using the biocompatible composite material as an implant and/or for body access.

16. The method according to claim 15, wherein the medical device is formed as a voice prosthesis.

17. The method according to claim 15, wherein the medical device is formed as a catheter or fistula adapter.

18. The biocompatible composite material according to claim 2, wherein the biocompatible elastomeric precursor material comprises an unvulcanized silicone layer.

19. The biocompatible composite material according to claim 13, wherein the medical device is a catheter.

20. The method according to claim 14, wherein the biocompatible elastomeric precursor material comprises an unvulcanized silicone.
